(19) United States
(12) Patent Application Publication
(10) Pub. No.: US 2004/0037792 A1
(43) Pub. Date: Feb. 26, 2004

(54) Deodorizing composition and method of using same

(75) Inventors: Tadahiro Hiramoto, Hiratsuka City (JP); Kenji Saiki, Hiratsuka City (JP); Yasutaka Mishima, Hiratsuka City (JP); Tetsuo Nakatsu, Chappaqua, NY (US)

Correspondence Address: DARBY & DARBY P.C., Post Office Box 5257, New York, NY, 10150-5257, US

(73) Assignee: TAKASAGO INTERNATIONAL CORP., Tokyo (JP)

(21) Appl. No.: 10/410,520

(22) Filed: Apr. 9, 2003

Related U.S. Application Data
(63) Continuation-in-part of application No. 09/637,076, filed on Aug. 11, 2000, now abandoned.

(51) Int. Cl. A61K 7/32

(52) U.S. Cl. 424/65

(57) ABSTRACT

A deodorant composition is provided including a phenolic compound, at least one enzyme capable of oxidizing the phenolic compound, and/or a deodorant synergy agent that is either a flavor and a fragrance. The fragrance or the flavor possesses a rating for freshness, cleanness and pleasurableness greater than 50 on a scale of from 1 to 100. Preferably, the fragrance is citrusy, fruity, floral (or flower), musky, minty, green, fresh, woody, sandal, spicy, flower and perilla-type accord. The flavor is preferably citrusy, pepperminty, fruity, juicy, minty, herby, vanilla, spicy, flower and perilla-type accord. A method of deodorizing a mal-odor is also provided which includes mixing the deodorant composition of the present invention with a suitable carrier to form a product for treating mal-odors and applying the product to either an article or a mammal.

### BACKGROUND OF THE INVENTION

This application is a continuation-in-part of U.S. patent application Ser. No. 09/637,076, filed Aug. 11, 2000.

The present invention relates to a deodorizing composition consisting of an enzyme substrate mixture (hereinafter referred to as “ESM”) which includes an oxidation substrate and/or an oxidative enzyme, and certain flavors or fragrances (hereinafter referred to as “FOF”). More specifically, the present invention relates to a deodorizing composition including a phenolic compound capable of acting as a deodorant, an enzyme capable of oxidizing phenolic compounds into compounds having a quinone structure, and FOF.

There are a number of deodorants presently available that are used to get rid of various mal-odors in the environment. Mal-odors are present in common nitrogen containing compounds such as ammonia, urea, indole, skatole and amines; sulfur containing compounds such as methyl mercaptan, hydrogen sulfide and dimethyl sulfide; and small fatty acids such as butyric acid. Of particular concern here are odors associated with halitosis (bad breath), body odor and bodily excretions including feces and urine. These odors are usually associated with production of volatile sulfur compounds such as methyl mercaptan and dimethyl sulfide and nitrogen containing compounds such as amines, ammonia and urea. Specifically, urine is a common source of ammonia and urea mal-odor while halitosis (bad breath) is a common source of sulfide mal-odors. These sources can cause detectable mal-odors at very low concentrations. The threshold concentration is the minimum level of detection of human olfactory senses below which the odor is undetectable. For sulfide compounds in particular, the odor threshold is very low.

Natural plant extracts have been identified as useful in controlling undesirable odors such as bad breath and body odor. For example, red beetroot, cacao beans, coffee beans and parsley extracts have been disclosed as useful deodorizers. See Japanese Laid-Open Publication No. 60-207664. Other plant extracts have also been found useful for this purpose, including persimmon extract disclosed in Japanese Laid-Open Publication No. 61-87562, and butterbur plant extract disclosed in Japanese Laid-Open Publication No. 61-206448.

The source of halitosis has often been associated with production of volatile sulfur compounds in the mouth related to breakdown of food by bacteria. Hydrogen sulfide and methyl mercaptan are suspected as the primary cause of the mal-odor. Dimethyl sulfide is also present. A number of naturally occurring plant extracts are known to be effective as deodorants against methyl mercaptan. Among these are rosmanol and camosol extracts from rosemary (Rosmalimus officinalis L.), and sage. An extract of thyme containing biphenyl compounds has similarly been found to be effective. Kayoko Miura et al., Structure and Activity of New Deodorant Biphenyl Compounds from Thyme (Thumus vulgaris L.), i Chem. Pharm. Bull., 37(7), pp. 1816-1819 (1989). In particular, extracts of thyme were studied and the data suggested the compounds were dimers of a hydroxy thymol derivative. Deodorant activity of thyme extracts against methyl mercaptan was found to be comparable to that of rosmanol and carnosol.

Certain marine algae have also been found to contain compounds that are effective deodorizers. Brown algae extracts that were effective in reducing the odor of methyl mercaptan were found to contain 1,3,5-trihydroxybenzene and trimers of phloroglucinol. Noriyoshi Kita et al., Screening test for deodorizing substances from marine algae and identification of phlorotannins as the effective ingredients in Eisenia bicyclis, J. of Applied Physiology, 155-162, 2, 1990.

Some naturally occurring plant extracts are also effective against nitrogen containing compounds. For example, extracts of sage, rosemary and thyme have been reported to suppress the trimethylamine odor.

Other known deodorants of plant origin include chlorophylls and certain flavonoids including catechins. In particular, the green tea catechins, the main components of green tea, have long been known to be effective against methyl mercaptan (CH₃SH). Among the green tea catechins, epigallocatechin gallate (EGCg) has the greatest deodorizing activity against methyl mercaptan. Other natural deodorizing substances have been found in plants, most of which are polyphenols and phenolic derivatives. It has been proposed that possible mechanisms of action of the deodorizing effect of polyphenols against methyl mercaptan involves hydrogen bonds between phenolic hydroxyl groups, physical association such as adsorption or an addition reaction. Deodorizing Mechanism of (−)-Epigallocatechin Gallate against Methyl Mercaptan, Hideyuki Yasuda and Tsutomu Arakawa, Biosci., Boitech, Biochem., vol 59(7), pp 1232-1236, 1995.

An investigation into possible mechanisms of action of tea catechins revealed that the molar intensity of the deodorizing activity of the various green tea catechins was in the same order as their anti-oxidative activity. The results of the study indicated that the deodorizing effect of EGCg against CH₃SH requires oxygen and that the reaction involved production of ortho-quinone from EGCg and then nucleophilic addition of the methylthio group to produce a thioether with the benzene ring. The study concluded that using EGCg in the oral cavity would result in the catechin being dissolved by saliva and captured by CH₃SH and the thiol-containing proteins and amino acids present in the mouth that act as substrates for oral bacteria.

One deodorizer using champignon mushroom extract has been shown to be effective not only against bad breath, but may also be used as a food additive to prevent odors produced from subsequent products of elimination. It is proposed that the deodorizer may be involved in metabolism to suppress these odors. U.S. Pat. No. 5,639,470 to Ishibashi et al. In addition, U.S. Pat. No. 5,639,470 discloses a deodorizer for use against bad breath as well as an ingredient in foods which produce bad breath or emit foul odors during preparation, such as methyl mercaptan. The product was also used as a food additive to effect a reduction of odor produced from subsequent urine and feces production after ingestion of mal-odorous products. The deodorizer includes a hydrophilic solvent extract of fruit bodies of champignon mushroom (Agaricus Bisporus) or powder of the extracts as an active component.

Artificially produced or synthetic deodorizers are also available. An alkyl or polyoxyalkylene ester of undecylenic acid has been used to deodorize mal-odorous animal feeds. U.S. Pat. No. 5,747,090. The deodorizing composition was found effective in reducing the foul odor from animal feeds when the feed contained from 0.1% to 5% by weight of the deodorizing compound.

The present inventors have previously discovered that the deodorizing effect of certain synthetic phenolic compounds is enhanced by combining the phenolic compound with a polyphenol oxidase. U.S. Pat. No. 5,804,170, herein incorporated by reference in its entirety. In addition, certain naturally occurring phenolic compounds will show the same enhanced performance when used in conjunction with an enzyme capable of oxidizing the phenol. See Japanese Laid-Open Patent No. 10-212221. An oral antiplaque composition containing certain synthetic noncationic phenols possessing antibacterial activity has also been disclosed. The composition includes a substantially water insoluble noncationic antibacterial phenol containing, relative to the hydroxyl group, an alkyl or cycloalkyl group, preferably tert-butyl in the 2 position. U.S. Pat. No. 5,912,274.

Numerous commercial products are available to reduce mal-odors, especially halitosis. Among these, antimicrobial rinses containing 0.2% of chlorhexidine gluconate have resulted in long term reductions in mal-odor. A common mouthwash, Listerine®, contains essential oils and alcohol, and provides long-lasting reduction in bad breath. Other mouthwashes are available that use zinc salts or quaternary ammonium compounds like cetylpyridinium chloride.

Although there are numerous deodorant compositions available, none are capable of completely eliminating odors or covering mal-odors.

FOF are also well known to cover mal-odor in environment. However, these are of limited use against strong odors. Usually the odor cannot be completely masked using even concentrated forms of FOF. In order to obtain sufficient cover of the odors, FOF must be used in concentrations that are undesirably strong. In other words, high concentrations of FOF are required to provide sufficient masking effects for the remaining mal-odors that are not quenched by chemical reactions or physical methods. Nevertheless such high concentration of FOF was used, the mal-odor cannot be covered, but the mal-odor is strengthened antagonistically depending on the type of FOF.

In this invention, the inventors found that certain FOF demonstrate synergy with certain deodorizing agents to completely eliminate mal-odor impression.

### OBJECTS AND SUMMARY OF THE INVENTION

Accordingly, it is an object of the present invention to provide a deodorant composition that has an excellent deodorizing effect and imparts a pleasant scent.

It is an object of the present invention to overcome the limitations of known deodorant compositions.

Briefly stated, the present invention is a deodorant composition including an enzyme substrate mixture (ESM) of individual substrate or enzyme capable of oxidizing the susbstrate, and of mixture of a substrate and an enzyme, in which certain FOF that work synergistically to neutralize undesirable odors was combined.

It is an object of the present invention to provide a deodorant composition including at least one phenolic compound, and/or at least one enzyme capable of oxidizing the phenolic compound(s), and a deodorant synergistic agent that is either FOF. The FOF are possesses a rating for freshness, cleanness and pleasurableness greater than 50 on a scale of from 1 to 100.

It is a further object of the present invention to provide a deodorant composition including at least one phenolic compound and/or at least one enzyme capable of oxidizing the phenolic compound(s), and a deodorant enhancing agent that is either FOF, where the fragrance is one of citrusy, fruity, floral (or flower), musky, moody, fresh, green, sandal, minty, spicy and herby accord fragrances, and the flavor is one of citrusy, minty, juicy, pepperminty, herby, fruity, vanilla, spicy, flower and perilla-type accord flavors.

It is a further object of the present invention to provide a method of deodorizing a mal-odor including mixing the deodorant composition of the present invention with a suitable carrier to form a product for treating mal-odors and applying the product to either an article or a mammal.

A deodorizing composition preferably can be used with known anti-microbial agents such as benzoic acid, sodium benzoate, chlorohexidine, triclosan, quarterly ammonium salts, thymol, and carvacrol, and/or anti-oxidant agents such as BHT, BHA, vitamin E and vitamin C to prevent deterioration of the deodorizing composition and maintain its deodorizing activity, and in addition, enhance suppression of the mal-odor generation by microbial putrefaction. A deodorizing composition preferably can be employed with known encapsulation technology to deliver the activity on demand.

The above, and other objects, features and advantages of the present invention will become apparent from the following description. It is to be understood that the invention is not to be limited in scope to the cited examples.

### DETAILED DESCRIPTION OF THE PREFERRED EMBODIMENT

An enzyme substrate mixture forms the deodorant composition of the present invention. The substrate or deodorant used in the present invention is a phenolic compound, preferably a polyphenolic compound. The phenolic compound may be derived from a naturally occurring source or may be made synthetically. For the natural sources, one or more extracts from a plant rich in phenolic compounds may be used as the substrate. For example, extracts from apple, banana, strawberry, apricot, peach, plum, citrus fruit, pineapple, pear, persimmon, cherry, papaya, mango, avocado, melon, loquat, fig, kiwi, prune, blueberry, black berry, raspberry, cranberry, black currant, mulberry, a rosemary, coffee bean, cacao bean, grape seed, grapefruit seed, pecan nut, cashew nut, chestnut, coconut, peanut, walnut, green tea, black tea, oolong tea, tobacco, olive leaves, olive fruit, mugwort, chrysanthemum, perilla leaf, garden thyme, sage, lavender, spearmint, peppermint, blessed thistle, hyssop, basil, a marigold, a dandelion, aloe, artichoke, German chamomile, agrimony, licorice, anise, yarrow, eucalyptus, wormwood, balm, angelica, fenugreek, paprika, fennel, anise seed, capsicum pepper, cardamom, celery seed, coriander seed, cumin seed, fennel seed, ginger, horseradish, marjoram, oregano, mustard, parsley, pepper, savory, tarragon, sesame, turmeric, wasabi, clove and dill seed, sunflower seed, fresh coffee bean, grape pericarp, grape seed, carrot, carrot leaf, burdock, tomato, broad bean, spinach, lotus root, eggplant, radish, turnip, beet, hop, chicory, onion, asparagus, green pepper, barley, wheat, corn, alfalfa, malt, soy bean, adzuki bean, green bean, French bean, mung bean, potato, sweet potato, sugar cane, yam taro and mushrooms.

The extract may be obtained from any method known in the art. Specifically, an extract may be obtained by mixing a plant source with a solvent followed by elimination of the solvent, if necessary. Examples of suitable solvents include water, alcohols, organic solvents and mixtures thereof. Preferably, the plant source will be pretreated by drying and cutting the plant into small pieces prior to the extraction process. Preferably the plant source will include rich phenolic compounds. Choice of the extraction solvent depends on the desired use of the deodorant composition. However, it is preferable to use a solvent that is safe for use in food products such as hot water, water containing ethanol and water containing propylene glycol.

The substrate may also be a synthetic phenolic compound having one or more phenolic hydroxyl groups. The term “phenolic hydroxyl group” used herein includes a hydroxyl group directly bonded to an aromatic ring such as the benzene ring. The aromatic ring may be any of benzene, pyridine, thiophene, naphthalene, biphenyl, and other aromatic rings having a structure that can be converted into ketones by oxidation of hydroxyl groups. Benzene is most preferred.

Examples of suitable phenolic compounds include diphenols such as catechol, 4-methylcatechol, 5-methylcatechol, resorcinol, 2-methylresorcinol, pyrogalol, 5-methylresorcinol, hydroquinone, 4,4′-biphenyldiol, 3,4′-diphenyldiol, dopa, dopamine, chlorogenic acid, caffeic acid, paracoumaric acid and tyrosine, gallic acid, tannic acid, epigallocatechin gallate, epicatechin gallate, epigallocatechin, epicatechin, catechin, ethyl protocatechuate, propyl gallate, butyl gallate, nordihydroguairetic acid, quercetin, quercitrin and rutin.

The amount of the phenolic compound is not limited. Generally, addition of from 1 to 100 mg of a phenolic compound is added per 100 mg of ESM. Preferably, from 10 to 100 mg is added per 100 mg of ESM. More preferably, from 20 to 80 mg is added per 100 mg of ESM.

The enzyme component of the ESM includes enzymes capable of oxidizing phenolic compounds, or a medium containing these enzymes. Preferred enzymes include enzymes capable of oxidizing the above-mentioned substrates into compounds having a quinone structure, enzymes capable of adding phenolic groups to the substrates and transforming the resulting product into a compound having a quinone structure, and a medium containing these enzymes.

Example of such enzymes include polyphenol oxidases, monophenol oxidases, catechol oxidases, peroxidases, oxidases forming hydrogen peroxidase and plant substances containg oxidative enzymes. More specifically, they include a phenolase, an urushiol oxidase, glucose oxidase, laccase, and tyrpsinase. Amongst them, preferred polyphenol oxidases include laccase, urushiol oxidase and phenolase. Alternatively, preferred monophenol oxidases include tyrosinase. Examples of the plants containing oxidative enzymes include at least one plant selected from the group consisting of fruits, vegetables, nuts, herbs, spices, teas, marine algae, bacteria and fungi. They also include mushrooms of genera Agaricus and Boletus such as Agaricus bisporus and Boletus pulverulentus. More specifically, such plants include apples, bananas, pears, strawberries, persimmons, pineapples, young citrus fruits, grapes, apricots, peaches, plums, papayas, quinces, avocados, mangos, cherries, melons, loquats, fligs, prunes, kiwis, blueberries, black berries, raspberries, cranberries, black currants, burdocks, eggplants, tomatoes, mugworts, lotus roots, lettuces, cabbages, beets, hops, parsnips, spinach, radishes, turnips, cauliflower, chicory, onions, celery, carrots, asparagus, green chili, horseradishes, wasabis, ginger, aloes, green peppers, barley, wheat, corns, alfalfa, malts, broad beans, soy beans, adzuki beans, red beans, green beans, sugar beans, French beans, mung beans, potatoes, sweet potatoes, sugar canes, yams, taros, tobaccos, olives, lacquer tree latex and chrysanthemums.

The prepared enzymes may be used alone or as a mixture. They are not limited to commercially available enzymes. They may be prepared according to a known method, such as enzyme-acetone powdering or lyophilization. The amount of enzymes to be added depends on its enzymatic activity, but should be sufficient to allow the enzyme to oxidize phenolic compounds. For the reaction to be accelerated the enzyme is preferably added in an amount that provides more than 100 units of enzyme activity per 100 mg of ESM. The above enzyme activity is defined as one unit, when the absorption value at OD 265 nm increases by 0.001 after substrate L-DOPA is oxidized at 25° C. and pH 6.5 for one minute.

From about 0.001% to about 90% enzyme by weight of the deodorant composition is usually sufficient. Preferably, from about 0.1% to about 20% enzyme is preferred. Most preferably, the enzyme is from about 0.1% to 10% of the deodorant composition.

The deodorant synergy agent component of the deodorizing composition is selected from a variety of FOF. For the FOF formulated in deodorant compositions, many fragrances and flavors are used. Preferably, the FOF are comprised of volatile compounds with the molecular weight of less than 500 Daltons, essential oils or extracts. Among them, the FOF are those having a score of more than 50 in fresh feeling, clean feeling and satisfaction rating, respectively, when the fresh feeling, clean feeling and satisfaction rating are represented by a numerical value (score) from 0 to maximum 100, respectively.

Additionally, the key perfumery component of the FOF does not contain a sulfur or nitrogen atom in the molecular structure. Examples of key perfumery components containing a sulfur or nitrogen atom are shown below.

Examples of the key perfumery component found in the flavor

| | Key perfumery component found |
|---|---|
| Flavors | in the flavor |
| Spring or Weise onion flavor | dipropyl disulfide |
| Chiboul flavor | dipropyl disulfide |
| Scallion flavor | dipropyl disulfide; allyl methyl disulfide |
| Leek flavor | Dimethyl disulfide; dimethyl trisulfide |
| Methyl mercaptan flavor | diallyl trisulfide; diallyl disulfide |
| Corn flavor | Dimethyl sulfide |
| Onion flavor | dipropyl disulfide; dipropyl trisulfide |
| Sesame flavor | Pyrazines |
| Crab flavor | Dimethyl sulfide |
| Cuttlefish flavor | Pyrazines |
| Shrimp or prawn flavor | Amines |
| Coffee flavor | furfuryl mercaptan |
| Mango flavor | Dimethyl sulfide |
| Passion fruit flavor | Oxathian |
| Cheese flavor | Dimethyl sulfide; methional |

The key perfumery component of the FOF as used herein means a component present in the fragrance, the flavor, the essential oil or the extract, which characterizes the odor of the fragrance or the flavor, and which is commonly used by those skilled in the art. Several methods are known for determining this component.

For example, the method can be employed where contribution degrees of the odors of the FOF components are calculated and the component of the FOF is determined from the calculated values. First, Odor activity value (OAV) of the FOF component P is calculated based on the following formula:

OAV=CA/CT

wherein the CA represents a concentration of the FOF component P in the whole aroma and the CT represents a threshold concentration of the FOF component P.

The OAV of all FOF components of the fragrance or the flavor are obtained. Subsequently, Odor contribution degree (%) (OCD) of FOF component P in the whole FOF is calculated based on the following formula:

OCD=(OAV/Toav)×100

wherein the OAV represents the OAV of the FOF component P and the Toav represents the sum of the OAV of all FOF components.

Then, the odor contribution degrees of all FOF components composing the fragrance or the flavor are calculated. The FOF component having the highest value is referred to as the key perfumery component of the FOF based on the odor contribution degrees obtained in this way.

Also, for example, FOF Extract Dilution Analysis Method (AEDA Method) is representative. More particularly describing, each diluted solution obtained by serially diluting (multiplicative dilution) the flavor or the fragrance with a solvent is smelled in the order from high to low concentrations, and is injected into a gas chromatography apparatus (GC-Olefactometry). Multiple FOF components in each diluted solution are detected , simultaneously the odor of each FOF component is smelled and it is recorded whether the odor is sensed or not. The odor of many FOF components composing the flavor or the fragrance goes out of sense in the range of definite dilution scales. However, the odor of minor FOF components is sensed even if diluted more highly than the above range. The odor goes out of sense when further diluted. At this rate of dilution, that is, at the rate of dilution just before the odor goes out of sense, the FOF component of which odor can be sensed is referred to as the key component of the FOF.

FOF are typically used in so-called blended perfumes by combining multiple FOF components, essential oils and extracts. The above extracts are referred to those obtained by extracting from plants containing perfumes by the standard method.

The preferred fragrance formulations are disclosed and hereinafter referred to as single impression or proper combination of “Citrusy” “Fruity”, “Floral (or flower)”, “Musky”, “Woody”, “Fresh”, “Green”, “Sandal”, “Minty”, “Spicy” and/or “Herby.” The amount of fragrance required depends on the application. Generally, the fragrance is added in an amount that will neutralize the odor of residual mal-odors which the ESM is unable to completely quench. Preferably, addition of from 0.01 to 50% of a fragrance is added per 100 mg of the deodorant composition. Preferably, the fragrance is from about 0.1% to about 30% of the deodorant composition. The preferred flavor formulations are disclosed and hereinafter referred to as single impression or combination of “Citrusy”, “Minty”, “Pepperminty”, “Herby”, “Fruity”, “Juicy”, “Vanilla”, “Spicy”, “Flower” and/or perilla-type.

The fragrance comprises at least one compound selected from the group consisting of Amyl salicylate, Benzyl acetone, Benzyl salicylate, 1,1,2,3,3-pentamethyl-2,3,5,6,7-pentahydroinden-4-one, 2,6,6,8-tetramethyltricyclo[5.3.1.0<1,5>]undecan-8-ol, Citronellol, tricyclo[5.2.1.0<2,6>]dec-4-en-8-yl acetate, tricyclo[5.2.1.0<2,6>]dec-4-en-8-yl propanoate, 2,6-dimethyloct-7-en-2-ol, 2,6-dimethylheptan-1-ol, Phenoxybenzene, 4,6,6,7,8,8-hexamethyl-6,7,8-trihydrocyclopenta[1,2-g]isochromane, Galbanum oil, Geranyl acetate, Geranyl nitrile, 3-ethoxy-1,1,5-trimethylcyclohexane, Hexyl cinnamic aldehyde, Hexyl salicylate, Iso-Bornyl acetate, 1-(3,4,10,10-tetramethylbicyclo[4.4.0]dec-5-en-3-yl)ethan-1-one, 2-((2Z)pent-2-enyl)-3-methylcyclopent-2-en-1-one, 3-[4-(tert-butyl)phenyl]-2-methylpropanal, Linalool, Methyl 2-aminobenzoate, (1E)-1-(6,6-dimethyl-2-methylenecyclohexyl)pent-1-en-3-one methyl 2-((1E)-1-aza-8-hydroxy-4,8-dimethylnon-1-enyl)benzoate, 2-nonynal dimethyl acetal, 2-phenylethan-1-ol, -Terpineol, 1-((6S,1R)-2,2,6-trimethylcyclohexyl)hexan-3-ol, 2-(tert-butyl)cyclohexyl acetate, 4-(tert-butyl)cyclohexyl acetate, 2-methoxynaphthalene, 1-(2,6,6,8-tetramethyltricyclo[5.3.1.0<1,5>]undec-8-en-9-yl)ethan-1-one, Acetyl iso-eugenol, Allyl amyl glycolate, 1,6,10,10-tetramethyl-5-oxatricyclo[7.4.0.0<2,6>]tridecane, (1S,2R,6R)-1,6,10,10-tetramethyl-5-oxatricyclo[7.4.0.0<2,6>]tridecane, Amyl -cinnamic aldehdye, Anisic aldehyde, Benzyl acetate, Bergamot oil, 7-methyl-2H,4H-benzo[b]1,4-dioxepin-3-one, Cinnamic alcohol, Citronellol, 2-methyl-3-[4-(methylethyl)phenyl]propanal, 2-oxabicyclo[4.4.0]decan-3-one, (2E)-1-(2,6,6-trimethylcyclohex-3-enyl)but-2-en-1-one, 2,6-dimethyloct-7-en-2-ol, 1,1-dimethyl-2-phenylethyl acetate, 2,6-dimethylheptan-1-ol, Eugenol, 2-oxacyclohexadecan-1-one, 3-(4-ethylphenyl)-2,2-dimethylpropanal, Geraniol, methyl 2-(3-oxo-2-pentylcyclopentyl)acetate, Hexyl acetate, Hexyl salicylate, 2H,4H,4aH,9aH-indano[2,1-d]1,3-dioxane, (3E)-4-(2,6,6-trimethylcyclohex-2-enyl)but-3-en-2-one, (3E)-4-(6,6-dimethyl-2-methylenecyclohexyl)but-3-en-2-enyl)-3-methylcyclopent-2-en-1-one, 1-(3,4,10,10-tetramethylbicyclo[4.4.0]dec-5-en-3-yl)ethan-1-one, 2-((2Z)pent-2-enyl)-3-methylcyclopent-2-en-1-one, 4-(4-hydroxy-4-methylpentyl)cyclohex-3-enecarbaldehyde, 3-[4-(tert-butyl)phenyl]-2-methylpropanal, (5E)-2,6-dimethylhept-5-enal, Methyl chavicol, (1E)-1-(6,6-dimethyl-2-methylenecyclohexyl)pent-1-en-3-one, 2,5-dioxacycloheptadecane-1,6-dione, Trans-2-Tridecenal, Phenyl ethyl acetate, Phenyl ethyl alcohol, Styrallyl acetate, Dimethyl cyclohexenal, 5-heptyl-3,4,5-trihydrofuran-2-one, 2-(tert-butyl)cyclohexyl acetate, -Fenchyl alcohol, 1-Decanal, 2,6-dimethyloct-7-en-2-ol, 4,6,6,7,8,8-Hexamethyl-6,7,8-trihydrocyclopenta[1,2-g]isochromane, Benzyl benzoate, Methyl 2-(3-oxo-2- pentylcyclopentyl)acetate, 3,7-Dimethyl-2,6-octadien-1-al, Linalyl acetate, Petigrain oil, Lemon oil, Lime oil, Geranyl nitrile, Tetrahydrolinalool, vanillin, caryophyllene. Lemon oil, Orange oil, citrus oil, cedarwood oil, Bois de rose oil, citronella oil, patchouli oil, eucalyptus oil, bay oil, grapefruit oil, mandarin oil, sndalwood oil, juniper berry oil, rose oil, ylang oil, tangerine oil, geranium oil and Limonene; and

The flavor comprises at least one compound selected from the group consisting of lemon oil, orange oil, grape fruit oil, mint oil, peppermint oil, menthol, iso-pulegole, eucaryptol, p-mentha-3,8-diol, vanilyl butyl ether, apricot oil, cassia oil, cinnamic aldehyde, allyl hexanoate, isoamyl acetate, amyl alcohol, anethole, benzaldehyde, benzyl acetate, isobutyl acetate, butyl butyrate, isobutyl butyrate, camphor, carvone, □-caryophyllene, cinnamaldehyde, cinnamyl alcohol, citral, citronellyl actate, cumin aldehyde, cymene, decalactone, decanal, diacetyl, ethyl acetoacetate, ethyl anthranilate, ethyl butylate, ethyl hexanoate, ethyl lactate, ethyl 2-methylbutylate, ethyl salicylate, ethyl vanillin, ethyl menthol, eugenol, iso-eugenol, furfural, furfuryl alcohol, geraniol, geranyl acetate, hexanal, hexenal, hexynyl acetate, hexyl alcohol, ionone, irone, limonene, linalool, linalyl acetate, maltol, menthol, menthone, methyl acetate, methyl anthranilate, methyl cinnmate, methyl salicylate, nerol, nerolidol, nonalactone, nonanal, octalactone, octanal, octanol, octenol , octyl acetate, phenyl acetate, phenetyl alcohol, pinenes, piperanal, propyl acetate, thymol, undecalactone, anise oil, anise star oil, basil oil, bay leaves west Indian oil, bois de rose oil, buchu leaves oil, cardamon seed oil, cassia bark oil, chmomile flower Roman oil, cinnamon bark oil, cinnamon leaf oil, citronella oil, clove bud oil, cognac green oil, coriander oil, cubebs oil, cumin oil, eucalyptus oil, fennel sweet oil, methyl mercaptan oil, ginger oil, grapefruit oil.

The amount of flavor required depends on the application. Generally, the flavor is added in an amount that will eliminate the odor of residual mal-odors which the deodorant is unable to completely quench. Preferably, addition of from 0.01 to 50% of a flavor is added per 100 mg of the deodorant composition. Preferably, the flavor is from about 0.1% to about 10% of the deodorant composition.

The fragrance and the flavor formulations are as follows:

Composition of Citrusy Fragrance

| Citrusy Fragrance Materials | Ratio (%) |
|---|---|
| -Fenchyl alcohol | 0.2 |
| 1-Decanal | 1.0 |
| Citronellol | 3.8 |
| 2,6-dimethyloct-7-en-2-ol | 3.4 |
| 4,6,6,7,8,8-Hexamethyl-6,7,8-trihydrocyclopenta[1,2- | 2.5 |
| g]isochromane | |
| Benzyl benzoate | 2.5 |
| Methyl 2-(3-oxo-2-pentylcyclopentyl)acetate | 1.0 |
| 3,7-Dimethyl-2,6-octadien-1-al | 32.0 |
| Linalyl acetate | 0.6 |
| Linalool | 2.0 |
| Styrallyl acetate | 2.0 |
| Petigrain oil | 0.2 |
| Geraniol | 10.0 |
| Lemon oil | 7.0 |
| Lime oil | 27.0 |
| Butylhydroxytoluene | 2.4 |
| Geranyl nitrile | 2.4 |

Composition of Fruity and Floral Fragrance

| Fruity Floral Fragrance Materials | Ratio (%) |
|---|---|
| 1-(2,6,6,8-tetramethyltricyclo[5.3.1.0<1,5>]undec-8-en-9- | 2.0 |
| yl)ethan-1-one | |
| Acetyl iso-eugenol | 1.0 |
| Allyl amyl glycolate | 1.0 |
| 1,6,10,10-tetramethyl-5-oxatricyclo[7.4.0.0<2,6>]tridecane | 1.0 |
| (1S,2R,6R)-1,6,10,10-tetramethyl-5- | 0.5 |
| oxatricyclo[7.4.0.0<2,6>]tridecane | |
| Amyl -cinnamic aldehdye | 1.0 |
| Anisic aldehyde | 1.0 |
| Benzyl acetate | 4.5 |
| Benzyl salicylate | 4.5 |
| Bergamot oil | 0.5 |
| 7-methyl-2H,4H-benzo[b]1,4-dioxepin-3-one | 0.5 |
| Cinnamic alcohol | 0.5 |
| Citronellol | 7.0 |
| 2-methyl-3-[4-(methylethyl)phenyl]propanal | 0.5 |
| 2-oxabicyclo[4.4.0]decan-3-one | 1.5 |
| (2E)-1-(2,6,6-trimethylcyclohex-3-enyl)but-2-en-1-one | 0.5 |
| 2,6-dimethyloct-7-en-2-ol | 1.0 |
| 1,1-dimethyl-2-phenylethyl acetate | 4.5 |
| 2,6-dimethylheptan-1-ol | 2.5 |
| Eugenol | 1.0 |
| 2-oxacyclohexadecan-1-one | 3.5 |
| 3-(4-ethylphenyl)-2,2-dimethylpropanal | 1.0 |
| Geraniol | 1.5 |
| methyl 2-(3-oxo-2-pentylcyclopentyl)acetate | 15.5 |
| Hexyl acetate | 0.5 |
| Hexyl salicylate | 5.0 |
| 2H,4H,4aH,9aH-indano[2,1-d]1,3-dioxane | 0.5 |
| (3E)-4-(2,6,6-trimethylcyclohex-2-enyl)but-3-en-2-one | 1.0 |
| (3E)-4-(6,6-dimethyl-2-methylenecyclohexyl)but-3-en-2-one | 1.5 |
| 1-(3,4,10,10-tetramethylbicyclo[4.4.0]dec-5-en-3-yl)ethan-1- | 1.0 |
| one | |
| 2-((2Z)pent-2-enyl)-3-methylcyclopent-2-en-1-one | 0.1 |
| 4-(4-hydroxy-4-methylpentyl)cyclohex-3-enecarbaldehyde | 3.0 |
| 3-[4-(tert-butyl)phenyl]-2-methylpropanal | 9.0 |
| Linalool | 2.3 |
| (5E)-2,6-dimethylhept-5-enal | 0.3 |
| Methyl chavicol | 0.5 |
| (1E)-1-(6,6-dimethyl-2-methylenecyclohexyl)pent-1-en-3-one | 4.0 |
| 2,5-dioxacycloheptadecane-1,6-dione | 3.5 |
| Trans-2-Tridecenal | 1.0 |
| Phenyl ethyl acetate | 0.5 |
| Phenyl ethyl alcohol | 5.0 |
| Styrallyl acetate | 0.5 |
| Dimethyl cyclohexenal | 0.3 |
| 5-heptyl-3,4,5-trihydrofuran-2-one | 1.0 |
| 2-(tert-butyl)cyclohexyl acetate | 1.5 |

Composition of Musky Woody Fragrance

| Musky Woody Fragrance Materials | Ratio (%) |
|---|---|
| Amyl salicylate | 3.0 |
| Benzyl acetone | 1.5 |
| Benzyl salicylate | 8.0 |
| 1,1,2,3,3-pentamethyl-2,3,5,6,7-pentahydroinden-4-one | 0.5 |
| 2,6,6,8-tetramethyltricyclo[5.3.1.0<1,5>]undecan-8-ol | 1.5 |
| Citronellol | 3.0 |
| tricyclo[5.2.1.0<2,6>]dec-4-en-8-yl acetate | 0.5 |
| tricyclo[5.2.1.0<2,6>]dec-4-en-8-yl propanoate | 1.5 |
| 2,6-dimethyloct-7-en-2-ol | 3.0 |
| 2,6-dimethylheptan-1-ol | 0.5 |
| Phenoxybenzene | 0.5 |
| Dipropylene glycol | 11.0 |
| 4,6,6,7,8,8-hexamethyl-6,7,8-trihydrocyclopenta[1,2- | 2.5 |
| g]isochromane | |
| Galbanum oil | 0.5 |
| Geranyl acetate | 1.0 |
| Geranyl nitrile | 0.5 |
| 3-ethoxy-1,1,5-trimethylcyclohexane | 0.5 |
| Hexyl cinnamic aldehyde | 17.0 |
| Hexyl salicylate | 12.0 |
| Iso-Bornyl acetate | 0.5 |
| 1-(3,4,10,10-tetramethylbicyclo[4.4.0]dec-5-en-3-yl)ethan- | 3.0 |
| 1-one | |
| 2-((2Z)pent-2-enyl)-3-methylcyclopent-2-en-1-one | 0.5 |
| 3-[4-(tert-butyl)phenyl]-2-methylpropanal | 3.5 |
| Linalool | 3.5 |
| methyl 2-aminobenzoate | 0.5 |
| (1E)-1-(6,6-dimethyl-2-methylenecyclohexyl)pent-1-en-3- | 4.0 |
| one | |
| methyl 2-((1E)-1-aza-8-hydroxy-4,8-dimethylnon-1- | 0.5 |
| enyl)benzoate | |
| 2-nonynal dimethyl acetal | 0.5 |
| 2-phenylethan-1-ol- | 3.0 |
| terpineol | 3.5 |
| 1-((6S,1R)-2,2,6-trimethylcyclohexyl)hexan-3-ol | 0.5 |
| 2-(tert-butyl)cyclohexyl acetate | 3.5 |
| 4-(tert-butyl)cyclohexyl acetate | 4.0 |
| 2-methoxynaphthalene | 0.5 |

Composition of Green Floral Fragrance

| Fragrance Materials | Ratio (%) |
|---|---|
| 1-Hydroxy citronellol | 2.0 |
| Undecylic aldehyde | 0.1 |
| Octyl aldehyde | 0.1 |
| Lauryl aldehyde | 0.1 |
| □-Undeca lactone | 0.5 |
| Anethole | 0.2 |
| (2E)-4-((1R)-2,2,3-trimethylcyclopent-3-enyl)- | 0.2 |
| 2-ethylbut-2-en-1-ol | |
| 1-Citronellol | 1.0 |
| p-Cresyl phenyl acetate | 0.3 |
| Tricyclodecenyl propionate | 4.0 |
| Dimethylbenzyl carbinyl acetate | 3.0 |
| Dimethylbenzyl carbinyl alcohol | 2.0 |
| Dimetol | 3.0 |
| Eugenol | 1.0 |
| (3E)-4-(6,6-dimethyl-2-methylenecyclohexyl)but-3-en-2-one | 4.0 |
| 4,6,6,7,8,8-hexamethyl-6,7,8- | 10.0 |
| trihydrocyclopenta[1,2-g]isochromane | |
| and its isomers in benzyl benzoate | |
| Geraniol | 2.0 |
| Hexylcinnamic aldehyde | 5.0 |
| Hexyl salicylate | 8.0 |
| 1-(3,4,10,10-tetramethylbicyclo[4.4.0]dec-5-en-3-yl)ethan- | 4.5 |
| 1-one and its isomers | |
| Lime oil | 1.0 |
| Teterahydro linalool | 1.0 |
| Tetrahydro mugol | 1.0 |
| Phenylethyl alcohol | 12.0 |
| 4-methyl-2-(2-methylprop-1-enyl)-2H-3,4,5,6-tetrahydropyran | 0.1 |
| Dipropylene glycol | 33.9 |
| Total | 100.0 |

Composition of Mint Fragrance

| Fragrance Material | Ratio (%) |
|---|---|
| Mint oil | 32.0 |
| 1-Menthol | 16.0 |
| Peppermint oil | 20.0 |
| □-Damascone | 0.2 |
| Isoamyl acetate | 1.0 |
| Lemon oil | 6.0 |
| Dipropylene glycol | 24.8 |
| Total | 100.0 |

Peppermint Accord Flavor

| Material | Weight % |
|---|---|
| Ethylsalicylate | 10.0 |
| Dried bonito oleoresin | 1.0 |
| 1-Menthol | 30.0 |
| Peppermint oil | 54.0 |
| Hakka hakuyu | 5.0 |
| Total | 100.0 |

Mint Accord Flavor

| Material | Weight % |
|---|---|
| Peppermint oil | 20.0 |
| 1-Menthol | 40.0 |
| Anethole | 6.0 |
| Lemon oil | 1.0 |
| Spearmint oil | 10.0 |
| 1-Carvone | 23.0 |
| Total | 100.0 |

Lemon Accord Flavor

| Material | Weight % |
|---|---|
| Anethole | 2.0 |
| Eucalyptus oil | 10.0 |
| Lemon oil | 1.0 |
| 1-Carvone | 25.0 |
| Ethanol | 27.0 |
| 1-Menthol | 25.0 |
| Spearmint oil | 10.0 |
| Total | 100.0 |

Mint Accord Powdery Flavor

| Material | Weight % |
|---|---|
| Peppermint oil | 10.0 |
| Arabic gum | 30.0 |
| Dextrin | 60.0 |
| Total | 100.0 |

Perilla Accord Flavor

| Material | Weight % |
|---|---|
| Perilla leaves oil | 10.0 |
| Fatty acid esters having a medium chain | 90.0 |
| length | |
| Total | 100.0 |

As mentioned supra, the deodorant composition of the invention is applied to malodorous objects or articles, as well as mammals or birds. Examples of the mammals include human, pets e.g. dogs, cats, birds, and domestic animals e.g. cows, pigs and horses. The malodorous objects may be malodorous mouths, sweat skin, a fridge odor, industrial wastes, kitchen refuse, garbage, or feces or animal droppings. They further include the foods generating special odors such as meats, fish and natto (fermented soy beans).

The applying methods are not limited to any specific means. Typically, the deodorant composition is formulated into a ready-to-spray preparation, and sprayed onto the malodorous objects by any spraying means.

The deodorant composition of the invention may be used directly onto the malodorous objects. However, it can also be mixed beforehand with foods or detergents, so that malodors can be prevented from leaking out.

Examples of the preferred products or articles, with which the inventive deodorant composition are used, include a food, a candy, a cookie, a lozenge, a chewing gum, a mouthwash, a toothpaste, an oral care product, a household cleaning product, a toilet cleaner, a wet towel, a bath room cleaner, a kitchen cleaner, a shoe box spray, a hair care product, a shampoo, a conditioner, a hair spray, a skin care product, a cosmetic, a toiletry, a personal care product, a professional strength sanitizing product, a diaper, a napkin, a pad, an air-freshener, a medicine, a garbage spray, an agricultural product, a pet food, a litter care product, and a pet product.

The deodorant composition may also be supplemented with additives. Examples of the preferred additives include a solvent, a glycol, a glyceride, a grain powder, a lipid, a polymer, a protein, a starch, a polysaceharide, a surfactant, a gum, a silica gel, a powder, and a capsule.

The deodorizing composition is preferably used with known antimicrobial agents such as benzoic acid, sodium benzoate, chlorhexidine, triclosan, quarterly ammonium salts, thymol, and carvacrol, and/or anti-oxidant agents such as BHT, BHA, vitamin E and vitamin C. In this manner, the deodorizing composition and its activity are prevented from deterioration. Moreover, these additives obviate malodors generated by microbial putrefaction. Further, the deodorizing composition is preferably formulated into a capsule by any known encapsulation technology, so that its activity can be delivered on demand. It is also possible to add thereto other commonly used additives.

### Organoleptic Evaluation of Fragrances and Flavors

FOF evaluations were performed on the five fragrance compositions and the four flavors by a panel of 3-5 panelists. FOF were evaluated for freshness, cleanness and pleasurableness. FOF were evaluated using a 0-100 scale according to the following ratings.

| Score | Evaluation Impression |
|---|---|
| 0 | Totally disagree |
| 25 | Disagree |
| 50 | Partially agree |
| 75 | Agree |
| 100 | Totally agree |

FOF were evaluated for freshness, cleanness and pleasurableness which matched very well with ESM.

Scales from 0 to 100 of the fresh feeling, the clean feeling and the satisfaction rating in the fragrance and the flavor are determined as follows. A bottle is filled with 1 g of a mixture of the fragrance or the flavor and ESM at a ratio of 1:1 by weight. Groups of 3 to 5 panelists sensually evaluate the odor at a mouth of the bottle mouth according to the following rating, and the average value of the results is calculated. These numerical values thus calculated represent the fresh feeling, the clean feeling and the satisfaction rating.

Ratings of between 50 and 100 are seemed satisfactory. Results are reported below in Tables.

### Organoleptic Evaluation of Deodorizing Effect

Deodorizing effect was evaluated by five panelists. The evaluation was scored using a 0-5 scale according to the following ratings.

| Score | Mal-odor Impression |
|---|---|
| 0 | Totally not detectable |
| 1 | Slightly detectable |
| 2 | Detectable |
| 3 | Obvious |
| 4 | Strong |
| 5 | Very strong |

Evaluation of Fragrances

| Fragrance | Freshness | Cleanness | Pleasurableness |
|---|---|---|---|
| Citrusy | 85 | 82 | 82 |
| Fruity Floral | 67 | 79 | 81 |
| Musky Woody | 71 | 75 | 75 |
| Green Floral | 68 | 76 | 81 |
| Minty | 91 | 85 | 79 |

Evaluation of Flavors

| | | | Pleasurableness |
|---|---|---|---|
| Flavor | Freshness Index | Cleanness Index | Index |
| Pepperminty | 78 | 78 | 76 |
| Minty | 80 | 88 | 79 |
| Lemony | 93 | 80 | 95 |
| Minty Powder | 80 | 88 | 74 |
| Perilla-Type | 85 | 85 | 85 |

The results of the FOF evaluations showed that all FOF tested were rated between 67 and 95, and were thus satisfactory from the standpoint of freshness, cleanness and pleasurableness. OOE in the tables stands for Out of Evaluation.

### Organoleptic Evaluation of Deodorizing Effect

Deodorizing effect was evaluated by five panelists. The evaluation was scored using a 0-5 scale according to the following ratings.

| Score | Mal-odor Impression |
|---|---|
| 0 | Totally Not Detectible |
| 1 | Slightly Detectible |
| 2 | Detectible |
| 3 | Obvious |
| 4 | Strong |
| 5 | Very Strong |

### Embodiment 1 (E1)

In this test, the ability of the deodorant composition in eliminating urine odor is evaluated. A deodorant composition was prepared as follows. The substrate phenols were obtained from raw coffee beans. The raw beans were ground by grinder and extracted with hot water at 85-95° C. for 2 hours. The extract was filtrated and dried to give the phenol substrate. The polyphenol concentration was approximately 30%.

The enzymes were obtained from the burdock. The burdock was freeze-dried and ground to give the enzyme powder. The similar active enzyme was as an acetone powder. The specific enzyme activity was approximately 70 units/mg.

ESM-1 prepared was 1:1 (w/w) mixture of the substrate and the enzymes.

### COMPARATIVE EXAMPLE 1 (CE1)

As a control, 10 μl of water and 10 ml of urine in the vial bottle was incubated with shaking for 15 min or 1 week at 25° C.

### COMPARATIVE EXAMPLE 2 (CE2)

The mixture of 10 ml of urine and 15 mg of ESM-1 (0.15%) was incubated with shaking for 15 min or 1 week at 25° C.

### COMPARATIVE EXAMPLE 3 (CE3)

The mixture of 10 ml of urine and 10 μl of the citrusy fragrance (0.1%) was incubated with shaking for 15 min or 1 week at 25° C.

### COMPARATIVE EXAMPLE 4 (CE4)

The mixture of 10 ml urine and citrusy fragrance 50 μl (0.5%) was incubated with shaking for 15 min or 1 week at 25° C.

### Embodiment 1 (E1)

The mixture of 10 ml urine, 15 mg of ESM-1 (0.15%) and citrusy fragrance 10 μl was incubated with shaking for 15 min or 1 week at 25° C.

The deodorant effect of the deodorant composition of Embodiment 1 and Comparative Examples 1-5 were evaluated organoleptically by 5 panelists.

15 minutes incubation:

| | | | | Mal-odor | |
|---|---|---|---|---|---|
| | Freshness | Cleanness | Pleasurableness | Impression | |
| Sample | Index | Index | Index | Index | Evaluation |
| CE-1(Urine) | OOE | OOE | OOE | 5.0 | Very strong urine odor |
| CE-2 (Urine + | OOE | OOE | OOE | 0.6 | Very weak urine odor |
| ESM-1) | | | | | impression. Remarkable |
| | | | | | urine odor was quenched |
| | | | | | but still enough to be |
| | | | | | deteced. |
| CE-3 (Urine + | 25 | 16 | 16 | 3.8 | At first, fragrance odor |
| Fragrance | | | | | and then strong urine |
| Lime 0.1%) | | | | | odor. No significant |
| | | | | | urine odor quenching |
| | | | | | effect. |
| CE-4 (Urine + | 25 | 16 | 16 | 3.2 | At first, fragrance odor |
| Fragrance | | | | | and then strong urine |
| Lime 0.5%) | | | | | odor. No significant |
| | | | | | urine odor quenching |
| | | | | | effect. |
| E-1 (Urine + | 85 | 82 | 82 | 0 | No urine odor |
| ESM-1 + | | | | | impression with orginal |
| Fragrance | | | | | fragrance intact. |
| Lime 0.1%) | | | | | |

OOE in the Tables means “Out of Evaluation.”

One Week Incubation

| | | | | Mal-odor | |
|---|---|---|---|---|---|
| | | | | (Urine) | |
| | Freshness | Cleanness | Pleasurableness | Impression | |
| Sample | Index | Index | Index | Index | Evaluation |
| CE-1(Urine) | OOE | OOE | OOE | 5.0 | Very strong urine odor |
| CE-2 (Urine + | OOE | OOE | OOE | 0.6 | Very weak urine odor |
| ESM-1) | | | | | impression. Remarkable |
| | | | | | urine odor was quenched |
| | | | | | but still enough to be |
| | | | | | deteced. |
| CE-3 (Urine + | 8 | 0 | 8 | 4.4 | At first, fragrance odor and |
| Fragrance | | | | | then strong urine odor. No |
| 0.1%) | | | | | significant urine odor |
| | | | | | quenching effect. |
| CE-4 (Urine + | 16 | 16 | 16 | 3.4 | At first, fragrance odor and |
| Fragrance | | | | | then strong urine odor. No |
| 0.5%) | | | | | significant urine odor |
| | | | | | quenching effect. |
| E-5 (Urine + | 85 | 82 | 82 | 0 | No urine odor impression |
| ESM-1 + | | | | | with orginal fragrance |
| Fragrance | | | | | intact. |
| 0.1%) | | | | | |

The results indicated that the deodorant composition containing citrusy fragrance and the instant ESM worked synergistically.

### Embodiment 2 (E-2)

In this test, the ability of the deodorant composition in eliminating urine odor from cat litter is evaluated. A deodorant composition was prepared as follows. The substrate phenols were obtained from commercially available rosemary extracts (manufactured by Tokyo Tanable Seiyaku Kabushiki Kaisha). The proportion of polyphenol compounds was about 6% by weight.

The enzymes were obtained from the banana. The banana was freeze-dried and ground to give the enzyme powder. Specific enzyme activity was 60 unit/mg.

ESM-2 prepared was 1:1 (w/w) mixture of the substrate and the enzymes.

### COMPARATIVE EXAMPLE 5 (CE5)

As a control, the mixture of 10 ml of water and cat urine in the cat litter was incubated for 1 hour at 34° C.

### COMPARATIVE EXAMPLE 6 (CE6)

The mixture of 10 ml of water and 15 mg of ESM-2 and cat urine in the cat litter was incubated for 1 hour at 34° C.

### COMPARATIVE EXAMPLE 7 (CE7)

The mixture of 10 ml of water and 10 μl (0.1%) of mint fragrance and cat urine in the cat litter was incubated for 1 hour at 34° C.

### Embodiment 2 (E-2)

The mixture of 10 ml of water and 10 μl (0.1%) of mint fragrance, 15 mg of ESM-2 and cat urine in the cat litter was incubated for 1 hour at 34° C.

Deodorizing Effect

| | | | | Mal-odor | |
|---|---|---|---|---|---|
| | | | | (Urine) | |
| | Freshness | Cleanness | Pleasurableness | Impression | |
| Sample | Index | Index | Index | Index | Evaluation |
| CE-5 (Urine) | OOE | OOE | OOE | 5 0 | Very strong urine odor |
| CE-6 (Urine + | OOE | OOE | OOE | 0.7 | Very weak urine odor |
| ESM-2) | | | | | impression. Remarkable |
| | | | | | urine odor was quenched |
| | | | | | but still enough to be |
| | | | | | detected. |
| CE-7 (Urine + | 40 | 28 | 35 | 4.0 | At first, fragrance odor and |
| Fragrance | | | | | then strong urine odor. No |
| 0.1%) | | | | | significant urine odor |
| | | | | | quenching or covering |
| | | | | | effect. |
| E-2 (Urine + | 91 | 85 | 79 | 0 | No urine odor impression |
| ESM-2 + | | | | | with orginal mint fragrance |
| Fragrance | | | | | intact. |
| 0.1%) | | | | | |

The results show that the odor of urine in cat litter is eliminated when using the deodorizing composition of the present invention. Using a combination of an ESM consisting of a polyphenolic deodorizing compound and an enzyme resulted in diminished urine odor. However, the odor still remained. Using just a fragrance did neutralize the urine odor, however the original mint fragrance was compromised.

### Embodiment 3 (E-3)

In this test, the ability of the deodorant composition to eliminate the bleach odor from laundry is evaluated. 10 cm×10 cm of clothes were soaked in hypochlorite bleach for 2 minutes and rinsed once with water. The clothes were washed with powder detergent and then rinsed.

The substrate phenols were obtained from commercially available sunflower seed extracts (manufactured by DaiNippon Ink Kagaku Kogyo Kabushiki Kaisha). The proportion of polyphenolic compounds in the phenol substrate was about 6% by weight.

The enzymes were obtained from the unripened apple. The acetone powder containing the instant enzyme of the unripened apples were prepared from sliced apples followed by eliminating acetone. Specific enzyme activity was approximately 70 units/mg.

ESM-3 prepared was 1:1 (w/w) mixture of the substrate and the enzymes.

Composition of Powder Detergent

| Materials | Weight % |
|---|---|
| Sodium C-12-C-18 pareih sulfate | 15.0 |
| Sodium carbonate | 15.0 |
| Sodium meta-silicate | 13.0 |
| Sodium citrate | 15.0 |
| Carboxy methyl cellulose | 2.0 |
| Sodium sulfate | 38.0 |
| Musky Woody Fragrance | 1.0 |
| ESM-3 | 1.0 |
| Total | 100.0 |

Composition of bleach

| Material | Weight % |
|---|---|
| Sodium hypochlorite | 6.0 |
| Cocoyl dimethyl amine oxide | 5.0 |
| Potassium pyrophosphate | 3.0 |
| Water | 86.0 |
| Total | 100.0 |

### COMPARATIVE EXAMPLE 8 (CE8)

A powder detergent composition was prepared without ESM-3 and fragrance.

### COMPARATIVE EXAMPLE 9 (CE9)

A powder detergent composition was prepared with 1% of ESM-3.

### COMPARATIVE EXAMPLE 10 (CE10)

A powder detergent composition was prepared with 1% of fragrance.

### Embodiment 3 (E3)

A powder detergent composition was prepared with 1% of ESM-3 and 1% of fragrance.

Deodorizing Effect

| | | | | Mal-odor | |
|---|---|---|---|---|---|
| Sample | Freshness | Cleanness | Pleasurableness | of Bleach | Evaluation |
| CE-8 | OOE | OOE | OOE | 4.7 | Very strong bleach odor |
| CE-9 (ESM- | 28 | 25 | 29 | 3.3 | Very weak bleach odor |
| 3) | | | | | impression. Remarkable |
| | | | | | bleach odor quenching effect. |
| CE-10 | 28 | 25 | 29 | 3.3 | At first, fragrance odor then |
| (Fragrance) | | | | | bleach odor impression. No |
| | | | | | significant bleach odor was |
| | | | | | quenched. |
| E-3 (ESM-3 | 71 | 75 | 75 | 0 | No bleach odor impression |
| and | | | | | with original fragrance intact. |
| Fragrance) | | | | | |

The results show that the odor of bleach in clothing is eliminated when washed with a detergent admixed with the deodorizing composition of the present invention. Using a combination of an ESM consisting of a polyphenolic deodorizing compound and an enzyme resulted in diminished bleach odor. However, the odor still remained. Using just a fragrance did neutralize the remaining chlorine odor, however the original Musky Woody fragrance was compromised.

### Embodiment 4 (E4)

In this test, the ability of the deodorant composition to neutralize a body-odor type smell is evaluated. A solution of butyric acid, 0.25% in water was mixed with scrapings from an antiperspirant stick. Butyric acid has a typical cheesy and sweat odor.

Composition of Antiperspirant

| Material | Weight % |
|---|---|
| PEG-7 glyceryl cocoate | 2.0 |
| Hydrogenated oil | 5.0 |
| Myristyl myristate | 15.0 |
| Cyclomethicone | 33.5 |
| Stearyl alcohol | 20.0 |
| Stearyl isononenoate | 3.0 |
| Aluminum chlorohydrate | 20.0 |
| Fruity Floral Fragrance | 0.5 |
| ESM-4 | 1.0 |
| Total | 100 |

The deodorant effect of the deodorant composition of Embodiment 4 and Comparative Examples 11-13 was evaluated organoleptically by 3 panelists.

The substrate phenols were obtained from grape pericarps as follows. The grape pericarps (strain Campbell) were added into ethanol, and stirred for 2 hours at 70° C. The water solution was filtered, and the filtrate was adsorbed on XAD-2 (manufactured by Organo Kabushiki Kaisha filled in a column. The column was washed with water, and eluted with methanol. The methanol eluate was then condensed to dryness, to yield grape pericarp extracts. The latter contained about 50% by weight of polyphenolic compounds.

The enzymes were obtained from commercially available tyrosinase obtained from mushrooms (product of Sigma Chemical Co.).

ESM-4 prepared was 1:1 (w/w) mixture of the substrate and the enzymes.

### COMPARATIVE EXAMPLE 11 (CE11)

An antiperspirant composition was prepared without ESM-4 and fragrance.

### COMPARATIVE EXAMPLE 12 (CE12)

An antiperspirant composition was prepared with 1% of ESM-4.

### COMPARATIVE EXAMPLE 13 (CE13)

An antiperspirant composition was prepared with 0.5% of fragrance.

### Embodiment 4 (E4)

An antiperspirant composition with 1% of ESM-4 and 0.5% of fragrance.

Deodorizing Effect

| | | | | Mal-odor | |
|---|---|---|---|---|---|
| Sample | Freshness | Cleanness | Pleasurableness | of Sweat | Evaluation |
| CE-11 | OOE | OOE | OOE | 5.0 | Very strong butyrc odor |
| CE-12 (ESM- | OOE | OOE | OOE | 1.7 | Very weak butyric acid odor |
| 4) | | | | | impression. Remarkable butyric |
| | | | | | acid odor quenching effect. |
| CE-13 | 13 | 18 | 21 | 3.3 | At first, fragrance odor then |
| (Fragrance) | | | | | butyric acid odor impression. No |
| | | | | | significant butyric acid odor was |
| | | | | | quenched. |
| E-4 (ESM-4 | 67 | 79 | 81 | 0 | No butyric acid odor impression |
| and | | | | | with original fragrance intact. |
| Fragrance) | | | | | |

The results show that the odor of remaining butyric acid after use of antiperspirant was neutralized when using the deodorizing composition of the present invention. Using a combination of an ESM-4 consisting of a polyphenolic deodorizing compound and an enzyme resulted in diminished sweat odor. However, the sweat odor and another different odor still remained. Using just a fragrance did eliminate the odor of butyric acid, however the original fruity floral fragrance was compromised.

### Embodiment 5 (E5)

A cold permanent wave product was prepared, and tested for its capacity of deodorizing a remaining permanent odor. 1.8 g of tress of false hair were dipped or soaked in 50 ml of first solution for 30 minutes. The first solution was prepared by adjusting a 6 wt % thioglycolic acid water solution to pH9.3 with ammonia water. The tress of false hair was wiped with papers, and washed once with 100 ml of water. The hair was dipped in 50 ml of second solution (5% potassium bromate water solution) for 20 minutes. The hair was wiped with papers, and dipped in 100 ml of ESM-5 containing shampoo solution for 5 minutes. The hair was wiped with papers, and washed once with 100 ml of water. The hair was wiped with papers, and subjected to evaluation test.

The deodorant effect of the deodorant composition of Embodiment 5 and Comparative Examples 14-16 was evaluated organoleptically by 3 panelists.

The substrate phenols were obtained by grinding burdocks. The ground burdocks were extracted with ethanol under reflux for 2 hours. The extracts were filtered, and the filtrate was condensed to dryness to obtain a burdock extract. The latter contained about 1.5% by weight of polyphenolic compounds

The enzymes were lacasses obtained from urushi (products of Sigma Chemical Co.).

ESM-5 prepared was 1:1 (w/w) mixture of the substrate and the enzymes.

Composition of shampoo after cold wave perm

| Material | Weight % |
|---|---|
| Sodium lauryl sulfate | 40.0 |
| Sodium cocoamphoacetate | 10.0 |
| Cocamide DEA | 2.0 |
| Butylene glycol | 2.0 |
| Citric acid | 0.35 |
| Sodium chloride | 0.1 |
| Methylparaben | 0.2 |
| Propylparaben | 0.1 |
| Tetrasodium EDTA | 0.1 |
| Green Floral Fragrance | 0.5 |
| ESM-5 | 2.0 |
| Purified water | Balance |
| Total | 100.0 |

### COMPARATIVE EXAMPLE 14 (CE14)

A cold perm shampoo composition was prepared without ESM-5 and fragrance.

### COMPARATIVE EXAMPLE 15 (CE15)

A cold penn shampoo composition was prepared with 2% of ESM-5.

### COMPARATIVE EXAMPLE 16 (CE16)

A cold penn shampoo composition was prepared with 0.5% of green floral fragrance.

### Embodiment 5 (E5)

A cold penn shampoo composition was prepared with 0.5% of green floral fragrance and 2.0% of ESM-5.

A cold penn composition with 2% of ESM-5 and 0.5% of fragrance.

| | | | | Mal-odor of | |
|---|---|---|---|---|---|
| Sample | Freshness | Cleanness | Pleasurableness | Perm Solution | Evaluation |
| CE-14 | OOE | OOE | OOE | 5.0 | Very strong perm odor |
| CE-15 (ESM- | OOE | OOE | OOE | 0.7 | Very weak cold perm odor |
| 5) | | | | | impression. Remarkable |
| | | | | | colde perm odor quenching |
| | | | | | effect. |
| CE-16 | 18 | 23 | 21 | 3.0 | At first, fragrance odor |
| (Fragrance) | | | | | then cold perm odor |
| | | | | | impression. No significant |
| | | | | | cold perm odor was |
| | | | | | quenched. |
| E5 | 68 | 76 | 81 | 0 | No cold perm odor |
| (ESM-5 and | | | | | impression with original |
| (Fragrance) | | | | | fragrance intact. |

The results show that the odor of unpleasant cold penn odor was eliminated when using the deodorizing composition of the present invention. Using a combination of an ESM-5 consisting of a polyphenolic deodorizing compound and an enzyme resulted in diminished cold perm odor. However, the cold pern odor and another different odor still remained. Using just a fragrance did neutralize the cold perm odor, however the original green floral fragrance was compromised.

### Embodiment 6 “Mouthwash”

The deodorant effect of the deodorant composition of Embodiment 6 and Comparative Examples 17-19 were evaluated organoleptically by three panelists.

The substrate phenols were obtained from green tea leaves. The dried green tea leaves were ground by grinder and extracted with hot water at 85-95° C. for 2 hours. The extract was filtrated and the filtrate were washed with hexane three times. The water layer was dried to give the phenol substrate. The polyphenol concentration was approximately 20%.

The enzymes were obtained from the unripened pear. The unripened pears were sliced, freeze-dried and ground to give the enzyme powder. Specific enzyme activity was approximately 60 units/mg.

ESM-6 prepared was 1:1 (w/w) mixture of the substrate and the enzymes.

Composition of Mouthwash

| Material | Weight % |
|---|---|
| Ethyl alcohol | 10.0 |
| Polyoxyethylene hydrogenated castor oil | 2.0 |
| Peppermint flavor | 0.5 |
| Saccarin Na | 0.02 |
| Glycerin | 10.0 |
| Na benzoate | 0.05 |
| FD & C color | Appropriately |
| ESM-6 | 2.0 |
| Purified water | Balance |
| Total | 100.0 |

10 mL of methyl mercaptan extract prepared from 4 g of methyl mercaptan and 1 L of water in 50 mL bottle and mouthwash solution 1 mL were mixed and shaked for 15 min at 25° C. in the 50 mL bottle. The solution was organoleptically evaluated according to the score index.

### COMPARATIVE EXAMPLE 17 (CE17)

A mouthwash test solution was prepared without ESM-6 and fragrance.

### COMPARATIVE EXAMPLE 18 (CE18)

A mouthwash test solution was prepared with 2% of ESM-6.

COMPARATIVE EXAMPLE 19 (CE19)

A mouthwash test solution was prepared with 0.5% of flavor.

### Embodiment 6 (E6)

A mouthwash test solution was prepared with 2% of ESM-6 and 0.5% of flavor.

Deodorizing Effect

| | | | | Mal- | |
|---|---|---|---|---|---|
| Sample | Freshness | Cleanness | Pleasurableness | odor | Evaluation |
| CE-17 | OOE | OOE | OOE | 5.0 | Very strong garlic odor. |
| CE-18 (ESM- | OOE | OOE | OOE | 0.6 | Very weak garlic odor |
| 6) | | | | | impression. Remarkable garlic |
| | | | | | quenching effect. |
| CE-19 | 26 | 21 | 20 | 2.4 | At first, pepperminty flavor |
| (Flavor) | | | | | impression and then garlic odor |
| | | | | | impression. No significant garlic |
| | | | | | odor was quenched. |
| E-6 | 78 | 78 | 76 | 0 | No garlic odor with original |
| (ESM-6 and | | | | | pepperminty flavor intact. |
| Flavor) | | | | | |

The results show that the odor of unpleasant methyl mercaptan odor was eliminated when using the deodorizing composition of the present invention. Using a combination of an ESM-6 consisting of a polyphenolic deodorizing compound and an enzyme resulted in diminished methyl mercaptan odor, but the methyl mercaptan odor and another different odor still remained. Using just a flavor did neutralize the remaining methyl mercaptan odor, however the original flavor was compromised.

### Embodiment 7 “Toothpaste”

The deodorant effect of the deodorant composition of Embodiment 7 and Comparative Examples 20-23 were evaluated organoleptically by four panelists. The substrate phenols were obtained from peppermint leaves. The dried peppermint leaves were ground by grinder and extracted with hot water at 85-95° C. for 2 hours. The extract was filtrated and the filtrate were washed with hexane three times. The water layer was dried to give the phenol substrate. The polyphenol concentration was approximately 20%.

The enzymes were obtained from prematurely harvested pears. The latter were cut into slices and the slices were freeze dried and ground to obtain enzyme powders. The specific enzyme activity was approximately 60 units/mg.

ESM-7 prepared was 1:1 (w/w) mixture of the substrate and the enzymes.

Composition of Toothpaste

| Material | Weight % |
|---|---|
| Dicalcium phosphate | 10.0 |
| Sodium lauryl sulfate | 2.0 |
| Sodium carboxymethylcellurose | 0.5 |
| Sodium saccharin | 0.02 |
| Sodium benzoate | 10.0 |
| Glycerine | Balance |
| Mint type flavor | 1.0 |
| ESM-7 | 0.05 |
| Total | 100.0 |

1) The subject rinsed the mouth with water.

2) 10 mL of 50 ppm mal-odor (methyl mercaptan) solution was administered into the mouth, and after one minute, the solution was spilled out.

3) The breath was collected into 5 L plastic bag.

4) The subject brushed the teeth for a minute with the sample toothpaste and rinsed with 25 mL of water twice.

5) The breath was collected into 5 L plastic bag.

6) The plastic bag was organoleptically evaluated by 4 panelists.

### COMPARATIVE EXAMPLE 20 (CE20)

The breath was collected without brushing.

### COMPARATIVE EXAMPLE 21 (CE21)

After brushing with toothpaste without ESM-7 and flavor, the breath was collected for the evaluation.

### COMPARATIVE EXAMPLE 22 (CE22)

After brushing with toothpaste including 0.05% of ESM-7, the breath was collected for the evaluation.

### COMPARATIVE EXAMPLE 23 (CE23)

After brushing with toothpaste including 1% of flavor, the breath was collected for the evaluation.

### Embodiment 7 (E-7)

After brushing with toothpaste including 1% of flavor and 0.05% of ESM-7, the breath was collected for the evaluation.

Deodorizing Effect

| Sample | Freshness | Cleanness | Pleasurableness | Mal-odor | Evaluation |
|---|---|---|---|---|---|
| CE-20 | OOE | OOE | OOE | 5.0 | Very strong methyl mercaptan |
| | | | | | odor impression. |
| CE-21 | OOE | OOE | OOE | 3.3 | Strong methyl mercaptan odor |
| | | | | | impression. |
| CE-22 | OOE | OOE | OOE | 0.5 | Very weak methyl mercaptan |
| (ESM-7) | | | | | odor impression. Remarkable |
| | | | | | methyl mercaptan odor |
| | | | | | quenching effect |
| CE-23 | 36 | 33 | 47 | 2.0 | At first, minty flavor impression |
| (Flavor) | | | | | and then methyl mercaptan odor |
| | | | | | impression. No significant |
| | | | | | methyl mercaptan odor was |
| | | | | | quenched |
| E-7 (ESM- | 80 | 88 | 79 | 0 | No methyl mercaptan odor with |
| 7 + Flavor) | | | | | original minty flavor intact. |

The results show that the odor of unpleasant methyl mercaptan odor was eliminated when using the deodorizing composition of the present invention. Using a combination of an ESM-7 consisting of a polyphenolic deodorizing compound and an enzyme resulted in diminished methyl mercaptan odor, but the methyl mercaptan odor were still remained. Using just a flavor did neutralize the methyl mercaptan odor, however the original flavor was compromised.

### Embodiment 8 “Tablet Candy”

The deodorant effect of the deodorant composition of Embodiment 8 and Comparative Examples 24-26 were evaluated organoleptically by 4 panelists.

The substrate phenol was apple extract (product of Nikka Whisky Kabushiki Kaisha). The substrate contained about 75 wt % by weight of polyphenolic compounds.

The enzymes were obtained from potatoes (strain “Baron”) by cutting the potatoes into slices, and freeze-drying and grinding the slices to obtain an enzyme powders. The specific activity of the powdered enzymes was about 30 units/mg.

ESM-8 prepared was 1:1 (w/w) mixture of the substrate and the enzymes.

Composition of Tablet Candy

| Material | Weight % |
|---|---|
| Starches | 96.7 |
| Mint type powdery flavor | 0.8 |
| Sucrose fatty acid ester | 0.5 |
| ESM-8 | 2.0 |
| Total | 100.0 |

1) The subject rinsed the mouth with water.

2) 10 mL of 50 ppm mal-odor (methyl mercaptan) solution was administered into the mouth, and after one minute, the solution was spilled out.

3) The breath was collected into 5 L plastic bag.

4) The subject was eating the candy for 10 minutes.

5) The breath was collected into 5 L plastic bag.

6) The plastic bag was organoleptically evaluated by 4 panelists.

### COMPARATIVE EXAMPLE 24 (CE24)

The breath was collected after eating the candy without ESM-2 and flavor.

### COMPARATIVE EXAMPLE 25 (CE25)

After eating the candy with ESM-8, the breath was collected for the evaluation.

### COMPARATIVE EXAMPLE 26 (CE26)

After eating the candy with the flavor, the breath was collected for the evaluation.

### Embodiment 8 (E-8)

After eating the candy with ESM-8 and the flavor, the breath was collected for the evaluation.

Deodorizing Effect

| Sample | Freshness | Cleanness | Pleasurableness | Mal-odor | Evaluation |
|---|---|---|---|---|---|
| CE-24 | OOE | OOE | OOE | 4.5 | Very strong methyl mercaptan |
| (without | | | | | odor impression. |
| ESM-2 and | | | | | |
| Flavor) | | | | | |
| CE-25 (with | OOE | OOE | OOE | 0.5 | Very weak methyl mercaptan |
| ESM-2) | | | | | odor impression. Remarkable |
| | | | | | methyl mercaptan odor |
| | | | | | quenching effect. |
| CE-26 | 33 | 38 | 32 | 2.8 | At first, mint flavor impression |
| (with Flavor) | | | | | and then methyl mercaptan |
| | | | | | odor impression. No significant |
| | | | | | methyl mercaptan odor was |
| | | | | | quenched |
| E-8 | 80 | 88 | 74 | 0 | No methyl mercaptan odor |
| (ESM-2 + Flavor) | | | | | impression with original fresh |
| | | | | | minty powder flavor intact. |

The results show that the odor of unpleasant methyl mercaptan odor was eliminated when using the deodorizing composition of the present invention. Using a combination of an ESM-8 consisting of a polyphenolic deodorizing compound and an enzyme resulted in diminished methyl mercaptan odor, but the methyl mercaptan odor were still remained. Using just a flavor did neutralize the remaining methyl mercaptan odor, however the original flavor was compromised.

### Embodiment 9 “Chewing Gum”

The deodorant effect of the deodorant composition of Embodiment 9 and Comparative Examples 27-29 were evaluated organoleptically by 5 panelists.

The substrate phenol was obtained by extracting dried olive leaves with methanol under reflux at 60° C. for 3 hours. The extracts were condensed to dryness, and dissolved in water. The water phase was filtered, and the filtrate was washed three times with hexane. The water phase was condensed and dried to yield the phenolic substrate. The substrate contained about 20% by weight of polyphenolic compounds.

The enzymes were obtained from eggplants by cutting the eggplants into slices, and freeze-drying and grinding the slices, to obtain an enzyme powder. The specific activity of the powdered enzymes was about 30 units/mg.

ESM-9 prepared was 1:1 (w/w) mixture of the substrate and the enzymes.

Composition of Chewing Gum

| Material | Weight % |
|---|---|
| Gum base | 21.0 |
| Sugar powder | 63.9 |
| Corn starch | 12.5 |
| Lemon type flavor | 1.0 |
| Acidifier | 0.6 |
| ESM-1 | 1.0 |
| Total | 100.0 |

1) The subject rinsed the mouth with water.

2) 10 mL of 50 ppm mal-odor (methyl mercaptan) solution was administered into the mouth, and after one minute, the solution was spilled out.

3) The breath was collected into 5 L plastic bag.

4) The subject chewed the gum for 10 minutes.

5) The breath was collected into 5 L plastic bag.

6) The plastic bag was organoleptically evaluated by 5 panelists.

### COMPARATIVE EXAMPLE 27 (CE27)

The breath was collected after chewing the gum without ESM-1 and flavor.

### COMPARATIVE EXAMPLE 28 (CE28)

After chewing the gum with ESM-9, the breath was collected for the evaluation.

### COMPARATIVE EXAMPLE 29 (CE29)

After chewing the gum with the flavor, the breath was collected for the evaluation.

### Embodiment 9 (E9)

After eating the gum with ESM-9 and the flavor, the breath was collected for the evaluation.

| Sample | Freshness | Cleanness | Pleasurableness | Mal-odor | Evaluation |
|---|---|---|---|---|---|
| CE-27(without | OOE | OOE | OOE | 4.2 | Very strong methyl mercaptan |
| ESM-1 and | | | | | odor impression. |
| Flavor) | | | | | |
| CE-28 (with | OOE | OOE | OOE | 0.4 | Very weak methyl mercaptan |
| ESM-1) | | | | | odor impression. Remarkable |
| | | | | | methyl mercaptan odor quenching |
| | | | | | effect. |
| CE-29 | 33 | 28 | 30 | 3.2 | At first lemony flavor impression |
| (with Flavor) | | | | | and then methyl mercaptan odor |
| | | | | | impression. No significant methyl |
| | | | | | mercaptan odor was quenched. |
| E-9 | 93 | 80 | 95 | 0 | No methyl mercaptan odor |
| (ESM-1 + Flavor) | | | | | impression with original fresh |
| | | | | | lemony flavor intact. |

The results show that the odor of unpleasant methyl mercaptan odor was eliminated when using the deodorizing composition of the present invention. Using a combination of an ESM-1 consisting of a polyphenolic deodorizing compound and an enzyme resulted in diminished methyl mercaptan odor, but the methyl mercaptan odor still remained. Using just a flavor did neutralize the methyl mercaptan odor; however, the original flavor was compromised.

### Embodiment 10 (E-10)

A phenolic substrate was obtained from commercially available grape seed extracts (products of Kikkoman Kabushiki Kaisba). The extracts contained about 26% by weight of polyphenolic compounds.

The enzymes were obtained by cutting yam into pieces, and freeze-drying and grinding the pieces, to yield an enzyme powder. The specific enzyme activity of the powder was about 30 units/mg.

ESM-10 prepared was 1:1 (w/w) mixture of the substrate and the enzymes.

Deodorant composition prepared was 1:1 (w/w) mixture of the ESM-10 and a perilla-type flavor.

### COMPARATIVE EXAMPLE 30 (CE30)

The sample contained no ESM-10 nor perrilia-type flavor.

### COMPARATIVE EXAMPLE 31 (CE34)

The sample contained ESM-10 but no flavor.

### COMPARATIVE EXAMPLE 32 (CE32)

The sample contained perilla-type flavor, but no ESM-10.

### Embodiment 10 (E-10)

The sample contained ESM-10 and perilla-type flavor.

Deodorizing effect

| | Fresh- | Clean- | Pleasurable- | Mal- | |
|---|---|---|---|---|---|
| Sample | ness | ness | ness | odor | Evaluation |
| CE30(without | OOE | OOE | OOE | 5.0 | Strong amine |
| ESM-10 and | | | | | odor |
| flavor | | | | | |
| CE31(with | OOE | OOE | OOE | 1.0 | No natto odor, |
| ESM-10) | | | | | but some |
| | | | | | pyrazine odor; |
| | | | | | strong natto |
| | | | | | deodorizing |
| | | | | | effect |
| CE32(with- | 33 | 28 | 30 | 3.2 | A weak but |
| Flavor) | | | | | perceptible |
| | | | | | natto odor |
| E10(ESM-10 + | 93 | 80 | 95 | 0 | No natto odor; |
| flavor | | | | | initial perilla- |
| | | | | | type flavor |
| | | | | | retained |

The results show unpleasant natto odor is efficiently obviated by the inventive deodorant composition. When only the sample ESM-10 is used, pyrazine odor, instead of natto odor, is felt. When only the perilla-type flavor is used, natto odor cannot be removed completely, and the initial perilla-type flavor loses some effect.

### Embodiment 11(Comparative Example) (CE-11)

In the present embodiment, some flavors containing sulfur atoms were studied, and their effect on the human olfactory sense was evaluated.

10 g of water containing 100 ppm of methyl mercaptan(sodium salt) were prepared in a 50 ml vial bottle. 50 μl of the coffee flavor (0.5 wt. %) and 15 mg of ESM-1 were further added into the vial bottle, and stirred for 15 minutes at 25° C. The coffee flavor contained furfuryl mercaptan as a key component of the FOF. The composition of the coffee flavor is given in the next table.

Compositon of Coffee flavor

| Flavor component | Weight % |
|---|---|
| Furfuryl mercaptan | 0.2 |
| Franeol | 0.3 |
| Cyclotene | 0.5 |
| Diacetyl | 0.6 |
| Furfural | 0.1 |
| Guaiacol | 0.5 |
| Isovaleric acid | 0.1 |
| Vanillin | 0.1 |
| Coffee extract | 50.0 |
| Water | 21.3 |
| Ethyl alcohol(95%) | 26.3 |
| Total | 100.0 |

The coffee flavor was evaluated by five panelists. The results of the evaluation are given in the next Table.

Evaluation of the Coffee flavor

| Sample | Freshness | Cleanness | Pleasurableness |
|---|---|---|---|
| Coffee flavor | 71 | 75 | 75 |

In the present study, a phenolic substrate was prepared according to the method described for Embodiment 1 (E1) using a grinder having a 5 mm mesh, to obtain a coffee extract. The extract contained about 30% by weight of polyphenolic compounds.

The enzymes were obtained from burdock by cutting the burdocks into slices, and freeze-drying and grinding the slices, to obtain an enzyme powder. The specific activity of the powdered enzymes was about 200 units/mg.

The deodorant effect of the deodorant composition of Embodiment 11 and Comparative Examples 33-35 were evaluated organoleptically by five panelists.

ESM-11 prepared was 1:1 (w/w) mixture of the substrate and the enzymes.

Deodorizing effect

| | Fresh- | Clean- | Pleasurable- | Mal- | |
|---|---|---|---|---|---|
| Sample | ness | ness | ness | odor | Evaluation |
| CE33 | OOE | OOE | OOE | 5.0 | Strong methyl |
| (without | | | | | mercptan odor |
| ESM-11 | | | | | |
| and | | | | | |
| flavor | | | | | |
| CE34 | OOE | OOE | OOE | 0.3 | Slight sulfur odor, |
| (with | | | | | Weak odor |
| ESM-11) | | | | | Original from |
| | | | | | ESM-1 |
| CE35 | 26 | 21 | 20 | 3.2 | Initially coffee |
| (with | | | | | flavor, then |
| flavor) | | | | | Strong methyl |
| | | | | | mercaptan Odor |
| E11 (with | 10 | 10 | 5 | 3.6 | Disintegrated |
| ESM-11 | | | | | weak coffee fla- |
| and | | | | | vor; methyl |
| flavor) | | | | | mercaptan odor |
| | | | | | identifiable |

### COMPARATIVE EXAMPLE 33 (CE33)

10 ml of water solution containing 100 ppm of methyl mercaptan(sodium salt) and 10 micro-l of water were added in a 50 ml vial bottle, and stirred for 15 minutes at 25° C.

### COMPARATIVE EXAMPLE 34 (CE34)

15 mg of ESM-11 and 10 ml of water solution containing 100 ppm of methyl mercaptan(sodium salt) were added in a 50 ml vial bottle, and stirred for 15 minutes at 25° C.

### COMPARATIVE EXAMPLE 35 (CE35)

50 μ-1 of coffee flavor and 10 ml of water solution containing 100 ppm of methyl mercaptan(sodium salt) were added in a 50 ml vial bottle, and stirred for 15 minutes at 25° C.

The results show the coffee flavor is disintegrated and methyl mercaptan odor is identifiable, even if the deodorant composition is added(CE35). This Embodiment showed the deodorant agent having no synergistic property.

Having described preferred embodiments of the invention with reference to the accompanying drawings, it is to be understood that the invention is not limited to those precise embodiments, and that various changes and modifications may be effected therein by one skilled in the art without departing from the scope or spirit of the invention as defined in the appended claims.

What is claimed is:

1. A composition possessing deodorant properties which consists essentially of:
a) at least one phenolic compound having one or more phenolic hydroxy groups;
b) at least one enzyme capable of oxidizing said phenolic compound into a compound having a quinone structure; and
c) at least one synergistic deodorant agent selected from the group consisting of a fragrance and a flavor, wherein said fragrance is selected from at least one of the group consisting of a Citrusy fragrance, a Fruity fragrance, a Floral (or flower) fragrance, a Musky fragrance, a Woody fragrance, a Fresh fragrance, a Green fragrance, a Sandal fragrance, a Minty fragrance, a Spring fragrance and an Herby fragrance;
and said flavor is selected from at least one of the group consisting of a Citrusy flavor, a Minty flavor, a Pepperminty flavor, an Herby flavor, a Juicy flavor, a Fruity flavor, a Vanilla flavor, a Spicy flavor, a Flower flavor and a Perilla-type flavor.

2. The composition of claim 1, wherein said fragrance or said flavor has a freshness, cleanness and pleasurableness value greater than 50 on a scale of from 1 to 100.

3. The composition of claim 1 wherein said fragrance or said flavor comprises volatile compounds having a molecular weight of less than 500 Dalton, essential oils or extracts.

4. The composition of claim 3 wherein said volatile compounds having a molecular weight less than 500 Dalton and do not comprise a sulfur atom a nitrogen atom.

5. The composition of claim 3 wherein said essential oils or extracts contain volatile compounds comprising a sulfur or nitrogen atom, provided that said essential oils or extracts are not a the key perfumery component, and the mixture of phenolic compounds and enzymes demonstrates synergistic properties.

6. The composition of claim 1, wherein said phenolic compound comprises from about 0.1% to about 50% by weight of said composition.

7. The composition of claim 1, wherein said enzyme comprises from about 0.01% to about 90% by weight of said composition.

8. The composition of claim 1, wherein said synergistic deodorant agent comprises from about 0.1% to about 10% by weight of said composition.

9. The composition of claim 1 wherein said phenolic compound comprises from about 1 to 10% by weight of said composition.

10. The composition of claim 1 wherein said enzyme comprises from about 0.1% to about 20% by weight of said composition.

11. The composition of claim 1, wherein said enzyme comprises from about 1% to about 10% by weight of said composition.

12. The composition of claim 1, wherein said fragrance comprises from about 0.01% to about 30% by weight of said composition.

13. The composition of claim 1, wherein said fragrance comprises from about 0.1% to about 10% by weight of said composition.

14. The composition of claim 1, wherein said flavor comprises from about 0.01% to about 30% by weight of said composition.

15. The composition of claim 1, wherein said flavor comprises from about 0.1% to about 10% by weight of said composition.

16. The composition of claim 1, wherein said enzyme is selected from the group consisting of a polyphenol oxidase, a tyrosinase, a xithanchin oxidase and a plant material containing oxidative enzymes.

17. The composition of claim 16, wherein said plant material is derived from at least one of a fruit, a vegetable, a nut, a herb, a spice, a tea, a seaweed, a bacterium and a fungi.

18. The composition of claim 17, wherein said plant material is derived from at least one of an apple, a banana, a pear, a strawberry, a persimmon, a pineapple, a young citrus fruit, a grape, an apricot, a peach, a plum, a papaya, a quince, an avocado, a mango, a cherry, an apricot, a melon, a loquat, a fig, a prune, a kiwi, a blueberry, a black berry, a raspberry, a cranberry, a black currant, a burdock, eggplant, a tomato, a mugwort, a lotus root, a lettuce, a cabbage, a beet, a hop, a parsnip, a spinach, a radish, a turnip, a cauliflower, a chicory, an onion, a celery, a carrot, an asparagus, a green chili, an eggplant, a horseradish, a wasabi, a ginger, an aloe, a green pepper, a barley, a wheat, a corn, an alfalfa, a malt, a broad bean, a soy bean, an adzuki bean, a red bean, a green bean, a sugar bean, a French bean, a mung bean, a potato, a sweet potato, a sugar cane, a yam, a taro, a tea, a tobacco, an olive, a lacquer tree latex, a chrysanthemun and mushrooms.

19. The composition of claim 1, wherein said phenolic compound is an extract from a member selected from the group consisting of a banana, a strawberry, an apricot, a peach, a plum, a citrus fruit, a pineapple, a pear, a persimmon, a cherry, a papaya, a mango, an avocado, a melon, a loquat, a fig, a kiwi, a prune, a blueberry, a black berry, a raspberry, a cranberry, a rosemary extract, a coffee bean, a cacao bean, a grape seed, a grapefruit seed, a pecan nut, a cashew nut, a chestnut, a coconut, a peanut, a walnut, a green tea, a black tea, an oolong tea, a tobacco, a perilla leave, garden thyme, sage, lavender, spearmint, peppermint, blessed thistle, hyssop, basil, a marigold, a dandelion, an artichoke, German chamomile, agrimony, licorice, anise, yarrow, a eucalyptus, a warmwood, a balm, angelica, fenugreek, paprika, fennel, anise seed, capsicum pepper, cardamom, celery seed, coriander seed, cumin seed, fennel seed, ginger, horseradish, marjoram, oregano, mustard, parsley, pepper, savory, tarragon, sesame, turmeric, wasabi, clove and dill seed, sunflower seed extract, fresh coffee bean extract, grape pericarp extract, grape seed extract, apple extract, carrot leaf extract, tea leaf extract, green tea extract, Chinese tea extract, peppermint leaf extract, catechol, 4-methylcatechol, 5-methylcatechol, resorcinol, 2-methylresorcinol, pyrogalol, 5-methylresorcinol, hydroquinone, 4,4′-biphenyldiol, 3,4′-diphenyldiol, dopa, dopamine, chlorogenic acid, caffeic acid, paracoumaric acid and tyrosine.

20. A method of deodorizing a mal-odor, comprising:
admixing at least one phenolic compound, at least one enzyme capable of oxidizing said phenolic compound, and at least one deodorant synergy agent selected from the group consisting of a floar and a fragrance to form a deodorant composition;
wherein said fragrance is selected from at least one of the group consisting of Citrusy, Fruity, Floral (or flower), Musky, Woody, Fresh, Green, Sandal, Minty, Spicy and Herby accord, and said flavor is selected from at least one of the group consisting of Citrusy, Minty, Pepperminty, Herby, Juicy, Fruity, Vanilla, Spicy, Flower and Perilla-type accord;
admixing said deodorant composition with a carrier to form a product for treating mal-odors; and
applying said product for treating mal-odors to an article or mammal in need thereof.

21. The method of claim 20, wherein said carrier is selected from the group consisting of a solvent, a glycol, a glyceride, a grain powder, a lipid, a polymer, a protein, a starch, a polysaccharide, a surfactant, a gum, a silica gel, a powder, and a capsule.

22. The method of claim 20, wherein said product for treating mal-odors is selected from the group consisting of a food, a candy, a cookie, a lozenge, a chewing gum, a mouthwash, a toothpaste, an oral care product, a household cleaning product, a toilet cleaner, a wet towel, a bath room cleaner, a kitchen cleaner, a shoe box spray, a hair care product, a shampoo, a conditioner, a hair spray, a skin care product, a cosmetic, a toiletry, a personal care product, a professional strength sanitizing product, a diaper, a napkin, a pad, an air-freshener, a medicine, a garbage spray, an agricultural product, a pet food, a litter care product, and a pet product.

23. A product for treating mal-odors, comprising the composition of claim 1.

24. The product of claim 21, which is selected from the group consisting of a food, a candy, a cookie, a lozenge, a chewing gum, a mouthwash, a toothpaste, an oral care product, a household cleaning product, a toilet cleaner, a wet towel, a bath room cleaner, a kitchen cleaner, a shoe box spray, a hair care product, a shampoo, a conditioner, a hair spray, a skin care product, a cosmetic, a toiletry, a personal care product, a professional strength sanitizing product, an air-freshener, a medicine, a pet food, a garbage spray, an agricultural product, a pet food, a litter care product, and a pet product.
